(19) United States
(12) Patent Application Publication
(10) Pub. No.: US 2008/0089951 A1
(43) Pub. Date: Apr. 17, 2008

(54) Method for Inhibiting Cancer Using Arsenic Trioxide

(75) Inventor: Yok-Lam Kwong, Hong Kong (HK)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: The University of Hong Kong

(21) Appl. No.: 11/871,057

(22) Filed: Oct. 11, 2007

Related U.S. Application Data
(63) Continuation-in-part of application No. 11/867,834, filed on Oct. 5, 2007.
(60) Provisional application No. 60/829,126, filed on Oct. 11, 2006.

(51) Int. Cl. A61K 33/36; A61P 35/00

(52) U.S. Cl. 424/623

(57) ABSTRACT

It has been discovered that As₂O₃ suppresses tyrosine kinase receptors, in particular EGFR and IL-6R, by targeting RTKs to lysosomes and/or proteasome for degradation. This is the basis for the discovery that cancers dependent on RTKs for signaling, proliferation, survival, metastasis and differentiation can be treated with As₂O₃, preferably oral As₂O₃. Representative cancers include EGFR and cytokine dependent cancers, for example, head and neck squamous sarcomas and multiple myelomas, respectively.

### CROSS-REFERENCE TO RELATED APPLICATIONS

This application claims priority to and benefit of U.S. Provisional Patent Application 60/829,126 filed on Oct. 11, 2006 and is a continuation-in-part of U.S. Ser. No. 11/867,834 filed Oct. 5, 2007, and where permitted, all of which are incorporated by reference in their entirety.

### FIELD OF INVENTION

This invention relates to methods of inhibiting certain cancers by affecting expression, translation, and biological activity of cancers expressing receptor tyrosine kinases using arsenic trioxide.

### BACKGROUND OF THE INVENTION

Receptor tyrosine kinases (RTK) are of pathogenic significance in many types of cancer. RTKs regulate multiple signaling pathways and contribute to cancer development, progression, proliferation, differentiation and metastasis, There are slightly fewer than 100 tyrosine kinases known. Derangement of many of these tyrosine kinases has been shown to lead to tumorigenesis in a large number of cancers, including leukemias, cancers of the head and neck, lung and breast. Therefore, targeting RTK would be an important therapeutic advance in cancer treatment. Representative cancers with RTK components include head and neck squamous cell carcinoma (HNSCC) and multiple myeloma.

Head and Neck Squamous Cell Carcinoma (HNSCC) and the RTK Epidermal Growth Factor Receptor.

Head and neck squamous cell carcinoma (HNSCC) is a common malignancy and a worldwide health problem (Jemal, et al., CA Cancer J. Clin., 55(1):10-30 (2005)). Despite recent advances in surgical and chemotherapeutic treatment, about 40% of patients will still develop incurable or disseminated diseases. Palliative systemic therapy with single or combination chemotherapy leads to a response in approximately 30% to 40% of cases. However, the response is often brief, and the median survival is only 6 to 9 months (Cohen, et al., J Clin Oncol., 22(9):1743-52 (2004)).

The epidermal growth factor receptor (EGFR), a transmembrane protein with tyrosine kinase activity, is expressed at high levels in HNSCC, with the degree of expression correlating negatively with clinical outcome (Grandis, et al., J Natl Cancer Inst., 90:824-32 (1998)). Aberrant signaling involving the EGFR plays an important pathogenetic role in HNSCC. Accordingly, EGFR represents a therapeutic target in HNSCC (Pomerantz and Grandis, Semin Oncol., 31(6):734-43 (2004)). At the extracellular level, EGFR can be blocked by monoclonal antibodies or toxin conjugates directed against the ligand binding site. The intracellular part of the EGFR can be inhibited by specific tyrosine kinase inhibitors. Antisense strategies against EGER or proteins involved in EGFR signaling have also been reported (Pomerantz and Grandis, Semin Oncol, 31(6):734-43 (2004)).

Because of the extensive biologic consequences of EGFR activation, EGFR signaling is tightly regulated. Binding of ECGF to EGFR results in the phosphorylation of several tyrosine residues in the intracellular portion of EGFR, including tyrosine 1045. Phosphorylated tyrosine 1045 becomes a docking site for Cbl, a protein that contains a tyrosine kinase binding domain that binds phosphorylated tyrosine, and a RING finger domain that mediates E3 ubiquitin ligase activity (Marmor and Yarden, Oncogene, 23(11):2057-70 (2004)). Binding of Cbl to EGFR leads to ubiquitination of intracellular lysine residues of the receptor through the attachment of multiple monoubiquitin moieties (Mosesson, et al., J Biol. Chem., 278(24):21323-6 (2003)). Ubiquitinated EGFR is endocytosed and targeted to degradation in lysosomes.

Arsenic trioxide (As₂O₃) is highly efficacious in the treatment of acute promyelocytic leukemia (APL), and has shown promise in other malignancies, notably myeloma (Kwong, Expert Opin Drug Saf, 3(6)-589-97 (2004)). Interestingly, one of the molecular mechanisms of As₂O₃ in APL is arsenic-augmented attachment of the ubiquitin-related peptide SUMO-1 to the leukemogenic PML/RARA protein, leading it to degrade in the proteosome (Lalemand-Breitenbach, et al., J Exp Med., 193(12):1361-71 (2001)). Therefore, As₂O₃ may act by targeting oncoproteins for enhanced degradation.

Multiple Myeloma and the Interleukin-6 Receptor (which Contains a Component Related to RTK).

Another malignancy, multiple myeloma (MM), is a clonal plasma cell neoplasm. Conventional treatment strategies including standard chemotherapy or high-dose therapy followed by autologous stem cell transplantation are not curative in most instances. The majority of patients eventually relapse with the development of chemoresistant diseases. With conventional chemotherapeutic regimens, the median survival rate of patients with MM is about 3-5 years (Catley, et al., Drug Resist Updat., 8(4):205-18 (2005)). Recent advances in the treatment of MM have focused on the development of molecular-based therapeutic agents.

A critical molecule involved in MM survival and proliferation is interleukin-6 (IL-6). IL-6 may be produced by MM cells in an autocrine fashion, and by bone marrow stromal cells. Furthermore, adhesion of MM cells to stromal cells, and other cytokines secreted by MM cells including Tumour Necrosis Factor-α, vascular endothelial growth factor and Transforming Growth Factor, can further augment IL-6 production (Van de Donk, et al., Leukemia, 19(12):2177-85 (2005)).

IL-6 signal transduction is mediated via the IL-6 receptor (IL-6R) complex, which comprises two subunits, IL-6Rα and gp130. IL-6Rα binds IL-6 but possesses no intrinsic activity. Formation of the IL-6/IL-6Rα complex recruits the gp130 subunit and induces gp130 homodimerization. The cytoplasmic region of gp130 is associated with Janus kinase (JAK), which is activated upon gp130 dimerization. Phosphorylation of gp130 by JAK on specific tyrosine residues exposes a docking site for signaling molecules that contain the Src-homology-2 (SH2) domain. The three major signaling cascades thus activated include the Janus kinase/signal transducer and activator of transcription 3 (JAK/STAT3) pathway, the Ras/mitogen-activated protein kinase (MAPK) pathway, and the phosphatidylinositol-3 kinase (PI3K)/Akt pathway. While the JAK/STAT3 pathway is implicated in MM cell survival, the Ras/MAPK and PI3K/Akt pathways are known to promote MM cell proliferation. In addition, JAK/STAT3 and PI3K/Akt signaling has been shown to protect MM cells from Fas- and dexamethasone-induced apoptosis (Cohen, et al., J Clin Oncol., 22(9):1743-52 (2004)). IL-6 also plays important role in MM bone disease by stimulating osteoclastogenic factors production and osteoclast formation (Roodman, Blood Cells Mol Dis., 32(2):290-2 (2004)).

Another cytokine implicated in MM pathogenesis is insulin-like growth factor-1 (IGF-1), a multifunctional protein produced by stromal and endothelial cells. IGF-1 signals through binding to the IGF-1 receptor (IGF-1R), which comprises α- and β-subunits (Tai, et al., Cancer Res., 63(18):5850-8 (2003)). The β-subunit, which possesses tyrosine kinase activity, is activated upon ligand binding. IGF-1 induces MM cell proliferation and survival independent of IL-6 via activation of the MAPK and PI3K/Akt signaling pathways (Ferlin, et al., Br J Haematol., 111(2):626-34 (2000); Qiang, et al., Blood, 99(11):4138-46 (2002)). It may collaborate with IL-6 by augmenting cell responsiveness to the latter cytokine (Abroun, et al., Blood, 103(6):2291-8 (2004)).

It is an object of this invention to provide agents and methods for treating cancers that are dependent on tyrosine receptor kinases, particularly EGFR and IL-6R for signaling, proliferation, survival, metastasis, or differentiation.

It is another object of this invention to provide methods, strategies, doses, and dosing schedules for the administration of As₂O₃ in the clinical inhibition of cancers dependent on tyrosine receptor kinases, particularly EGFR and IL-6R for signaling, proliferation, survival, metastasis, or differentiation.

### SUMMARY OF THE INVENTION

It has been discovered that As₂O₃ suppresses tyrosine kinase receptors, in particular EGFR and IL-6R, by targeting RTKs to lysosomes and/or proteasome for degradation. This is the basis for the discovery that cancers dependent on RTKs for signaling, proliferation, survival, metastasis and differentiation can be treated with As₂O₃, preferably oral As₂O₃. Representative cancers include EGFR and cytokine dependent cancers, for example, head and neck squamous sarcomas and multiple myelomas, respectively.

### BRIEF DESCRIPTION OF DRAWINGS

FIG. 1A is a line graph of percent proliferating HNSCC FaDu cells versus concentration of As₂O₃ (IM) incubated for two days. FIG. 1B is a bar graph of caspase activity (arbitrary units) versus concentration of As₂O₃ (μM).

FIG. 2A is a bar graph of EGFR expression determined by Western blot analysis (arbitrary units) versus concentration of As₂O₃ (μM) in HNSCC FaDu cells incubated for a period of two days. FIG. 2B is a bar graph of constitutive phosphorylation of STAT3 determined by Western blot analysis (arbitrary units) versus concentration of As₂O₃ (μM) in HNSCC FaDu cells incubated for a period of two days. FIG. 2C is a bar graph of constitutive phosphorylation of STAT3 determined by Western blot analysis (arbitrary units) (arbitrary units) as a function of control or 10 μM anti-7EGFR antibody in HNSCC FaDu cells incubated for a period of two days. FIG. 2D is a bar graph of percent proliferating HNSCC FaDu cells versus control, 2.5 μM As₂O₃, or 10 μM anti-EGFR antibody incubated for two days.

FIG. 3 is a bar graph of percent expression of EGFR mRNA determinative quantitative reverse transcription polymerase chain reaction versus control or 2.5 μM As₂O₃ in HNSCC FaDu cells incubated for a period of two days.

FIG. 4 is a bar graph of As₂O₃-induced down-regulation of EGFR determined by Western blot analysis (percent control) versus concentration of NH₄Cl pretreatment of HNSCC FaDu cells incubated for a period of two days in 2.5 μM As₂O₃.

FIG. 5 is a bar graph of percent proliferating HNSCC FaDu cells versus DMSO or UO126 in HNSCC FaDu cells incubated in 2.5 μM As₂O₃ for two days.

FIG. 6A is a line graph of percent proliferating cells versus concentration of As₂O₃ (μM) in U266 myeloma cells incubated for 24 hours (▴), 48 hours (▪), or 72 hours (●). FIG. 6B is a line graph of percent proliferating cells versus concentration of As₂O₃ (μM) in U266 mycloma cells incubated with Z-VAD-FMK (▪), or control (●). FIG. 6C is a bar graph of percent stimulation of control versus concentration of As₂O₃ (μM) in U266 cells treated with interleukin-6 (IL-6) or insulin-like growth factor 1 (IGF-1).

FIG. 7A is a bar graph of STAT-3 phosphorylation determined by Western blot analysis versus incubation of U266 cells with IL-6 (50 ng/ml) in the presence or absence of 4 μM As₂O₃. FIG. 7B is a bar graph of Akt phosphorylation determined by Western blot analysis versus incubation of U266 cells with IL-6 (50 ng/ml) in the presence or absence of 4 μM As₂O₃. FIG. 7C of Erk1/2 phosphorylation determined by Western blot analysis versus incubation of U266 cells with IL-6 (50 ng/ml) in the presence or absence of 4 μM As₂O₃.

FIG. 8 shows IL-6 internalization determined by Western blot analysis versus time in hours in U266 cells transfected with pcDNA3.1-IL-6Rα (U266-IL-6Rα) versus log signal in the presence of 4 μM As₂O₃.

FIG. 9A is a line graph of down regulation of gp130 determined by band density in Western blots versus time in hours in U266 cells treated 4 μM As₂O₃ with or without 1 hour pre-treatment with the lysosomal inhibitor ammonium chloride (NH₄Cl, 40 mM). FIG. 9B is a line graph of down regulation of IL-6α determined by band density in Western blots versus time in hours in U266 cells treated with 4 μM As₂O₃ with or without 1 hour pre-treatment with the lysosomal inhibitor ammonium chloride (NH₄Cl, 40 mM). FIG. 9C is a line graph of down regulation of gp 130 determined by band density in Western blots versus time in hours in U266 cells treated 4 μM As₂O₃ with or without 1 hour pre-treatment with MB-132. FIG. 9D is a line graph of down regulation of IL-6α determined by band density in Western blots versus time in hours in U266 cells treated with 4 μM As₂O₃ with or without 1 hour pre-treatment with MB-132. FIG. 9E is a line graph of down regulation of gp130 determined by band density in Western blots versus time in hours in U266 cells treated 4 μM As₂O₃ with or without 1 hour pre-treatment with the pan-caspase inhibitor Z-VAD-FMK. FIG. 9F is a line graph of down regulation of IL-6α determined by band density in Western blots versus time in hours in U266 cells treated 4 μM As₂O₃ with or without 1 hour pre-treatment with the pan-caspase inhibitor Z-VAD-FMK.

### DETAILED DESCRIPTION OF THE PREFERRED EMBODIMENTS

I. Arsenic Trioxide Formulations

Arsenic trioxide is a standard treatment for acute promyelocytic leukemia. Studies in vitro have shown that As₂O₃ may be effective in MM. As₂O₃ induces MM cell apoptosis by manipulating the cellular redox state, leading to decreased intracellular GSH levels, disruption of mitochondria potential and cleavage of caspases 3, 8, 9 (Gartenhaus, et al., Clin Cancer Res., 8(2):566-72 (2002); Liu, et al., Blood, 101(103):4078-87 (2003)). As₂O₃-induced apoptosis may also be associated with the TNF-related apoptosis inducing ligand (APO/TRAIL) pathway, which induces caspase 8-dependent apoptosis (Akay and Gazitt, Cell Cycle, 2(4):358-68 (2003)). Furthermore, As₂O₃ induces G1/G2 cell cycle arrest by upregulating the expression of the cyclin-dependent kinase (CDK) inhibitor p21, with concomitant increase in p21 binding to and hence inhibition of cell cycle-regulatory proteins CDK6, cyclin A and cyclin E, resulting in inhibition of the activities of these proteins (Park, et al., Cancer Res., 60(11):3065-71 (2000)).

In preliminary clinical studies, As₂O₃ has also shown significant activities in MM. In a phase II study involving 14 patients with relapsed and refractory MM, response was observed in three patients, with prolonged stable disease observed in a fourth patient (Munshi, Oncologist, 6 Suppl 2:17-21 (2001)). In another multicenter, phase II trial of 24 similar patients, As₂O₃ achieved a response rate of 33%, and stable disease in an additional 25% of patients (Hussein, et al., Br J Haematol., 125(4):470-6 (2004)). Furthermore, satisfactory response rates were also observed in trials using As₂O₃ in combination with ascorbic acid or melphalan (Bahlis, et al., Clin Cancer Res., 8(12):3658-68 (2002); Berenson, et al., Clin Lymphoma, 5(2):130-4 (2004)).

It has been shown previously that oral arsenic trioxide can be successfully formulated for the treatment of acute promyelocytic leukemia (Kumana, et al., Eur J Clin Pharmacol., 58(8):521-6 (2002)). Furthermore, oral arsenic trioxide is highly efficacious for this leukemia and results in very few side effects (Siu, et al., Blood, 108(1):103-6 (2006)). Recently, oral arsenic trioxide has been shown to produce minimal QT prolongation in the heart, meaning that it is very safe for prolonged use (Siu, et al., Blood, 108(1):103-6 (2006)). For the treatment of other cancers, it is expected that prolonged use of arsenic trioxide is needed. Therefore, oral arsenic trioxide is preferred for treatment of patients.

Arsenic Trioxide

Arsenic trioxide is available from a number of different suppliers. Arsenic trioxide is an amphoteric oxide which is known for its acidic properties. It dissolves readily in alkaline solutions to give arsenites. It is much less soluble in acids, but will dissolve in hydrochloric acid to give arsenic trichloride or related species. It reacts with oxidizing agents such as ozone, hydrogen peroxide and nitric acid to give arsenic pentoxide, As₂O₅. It is also readily reduced to arsenic, and arsine (AsH₃) may also be formed.

Arsenic trioxide has many uses including as: a starting material for arsenic-based pesticides; a starting material for arsenic-based pharmaceuticals, such as a neosalvarsan, a synthetic organoarsenic antibiotic; a decolorizing agent for glasses and enamels, a wood preservative, and a cytostatic in the treatment of refractory promyelocytic (M3) subtype of acute myeloid leukemia.

An oral arsenic trioxide (As₂O₃) is highly efficacious for relapsed acute promyelocytic leukemia. Oral As₂O₃ causes a smaller prolongation of QT intervals, and therefore is a much safer drug for treating leukemia.

Formulations

The following delivery systems, which employ a number of routinely used pharmaceutical carriers, are only representative of the many embodiments envisioned for administering the instant compositions.

Parenteral Formulations

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Enteral Formulations

Oral delivery systems include solid dosage forms such as tablets (e.g, compressed tablets, sugar-coated tablets, film-coated tablets, and enteric coated tablets), capsules (e.g., hard or soft gelatin or non-gelatin capsules), blisters, and cachets. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). The solid dosage forms can be coated using coatings and techniques well known in the art.

Oral liquid dosage forms include solutions, syrups, suspensions, emulsions, elixirs (e.g., hydroalcoholic solutions), and powders for reconstitutable delivery systems. The formulations can contain one or more carriers or excipients, such as suspending agents (e.g., gums, zanthans, cellulosics and sugars), humectants (e.g., sorbitol), solubilizers (e.g., ethanol, water, PEG, glycerin, and propylene glycol), surfactants (e.g., sodium lauryl sulfate, Spans, Tweens, and cetyl pyridine), emulsifiers, preservatives and antioxidants (e.g., parabens, vitamins E and C, and ascorbic acid), anti-caking agents, coating agents, chelating agents (e.g., EDTA), flavorants, colorants, and combinations thereof. The compositions can be formulated as a food or beverage (e.g., a shake) containing buffer salts, flavoring agents, coloring agents, sweetening agents, and combinations thereof.

II. Methods of Treatment

Tyrosine kinase receptors play an important role in multiple signaling pathways and contribute to cancer development, progression, proliferation, differentiation and metastasis. Representative cancers that depend on TKRs include, but are not limited to, head and neck squamous cell carcinoma (HNSCC) and multiple myeloma. Representative TKRs that are involved in cancer include, but are not limited to, EGFR and IL 6R (which contains a component related to RTK). In the method described herein, an effective amount of arsenic trioxide to downregulate the TKR in a cancer cell is administered to an individual for treating cancers that are dependent on TKRs.

A. EGFR Dependent Cancers

Owing to the protean biologic consequences of EGFR signaling in cellular proliferation, differentiation, survival, angiogenesis and inhibition of apoptosis, suppression of EGFR holds much promise in the control of EGFR-dependent malignancies. Currently available strategies offer a two-prong extracellular\intracellular approach (Choong and Cohen, Crit Rev Oncol Hematol., 57(1):25-43 (2006)). The extracellular domain of EGFR can be blocked by anti-EGFR antibodies, which prevent ligand binding to EGFR. Antibody-dependent cellular cytotoxicity may also be involved. Molecular determinants control the sensitivity to anti-EGFR antibodies, including the level of EGFR expression that correlate positively with efficacy. However, anti-EGFR antibodies possess relatively poor clinical efficacy when used as single agents, with a response rate of less that 10%. The intracellular EGFR tyrosine kinase domain of EGFR can be blocked by small-molecule tyrosine kinase inhibitors (TKI). Administered singly, TKIs showed a response rate varying between 10-20%. The response is correlated with somatic mutations in the receptor kinase domain and/or EGFR gene amplification (Jane, Semin Oncol., 32(6 Suppl 10):S9-15 (2005)) implying that the efficacy of TKI is limited in cancers with wildtype EGFR.

The data presented in the Examples show that the EGFR signaling pathway was constitutively activated in the HNSCC line FaDu. FaDu was critically dependent on EGFR signaling for cellular survival and proliferation, as treatment with a blocking EGFR antibody led to apoptosis. As₂O₃ treatment also resulted in down-regulation of EGFR, which produced biologic effects mimicking those caused by antibody blockade of EGFR.

EGF gene transcription was active in FaDu cells. As₂O₃ treatment did not affect EGF transcription, ruling out disruption of EGFR signaling owing to down-regulation of EGF. Therefore, As₂O₃ targeted EGFR but not EGF in the EGFR signaling pathway, suggesting that EGFR down-regulation played an important part in mediating cytotoxicity.

The data further demonstrated that EGFR down-regulation occurred at a post-transcriptional level. EGFR gene transcription was unaffected by As₂O₃, as shown by semi-quantitative and quantitative polymerase chain reaction. These results might be expected, as arsenic is not known to directly bind DNA or transcription factors. Therefore, As₂O₃ likely down-regulates EGFR by increasing its degradation.

Normal ligand dependent degradation of EGFR starts with stimulation by EGF, leading to EGFR phosphorylation at tyrosine 1045 (Marmor and Yarden, Oncogene, 23(11):2057-70 (2004)). The data show that As₂O₃ also mediated an apparent EGF independent EGFR phosphorylation at tyrosine 1045. Once this had occurred, the molecular consequences were similar to those due to ligation of EGF. Cbl was recruited to phosphorylated tyrosine 1045. By virtue of its RING figure domain that mediated E3 ubiquitin ligase activity, Cbl promoted EGFR mono-ubiquitination, targeting EGFR to lysosomal degradation (Mosesson, et al., J Biol. Chem., 278(24):21323-6 (2003)).

EGFR is known to be involved in signaling networks activated by stimuli that are not ligands of the receptor. Participation in these signaling pathways also entails EGFR phosphorylation similar to that induced by EGF. Non-EOF stimuli that activate EGPR include specific agonists of other membrane receptors, membrane depolarizing agents, and environmental stressors (Carpenter, J Cell Biol, 146(4):697-702 (1999)). As₂O₃ falls into the last category, as previous studies have shown that As₂O₃ induces many stress proteins (Del Razo, et al., Toxicol Appl Pharmacol., 177(2):132-48 (2001)). Important stress proteins include heat shock protein and the non-tyrosine kinase protein Src. Interestingly, arsenic-induced Src expression leading to phosphorylation of downstream target proteins has been well recognized. Src is in fact known to phosphorylate EGFR at all five sites that autophosphorylate upon stimulation by EGF (Hubbard and Till, Annu Rev Biochem., 69:373-98 (2000); Simeonova, et al., J Biol. Chem., 277(4):2945-50 (2002)). The data disclosed here showed that Src was activated by As₂O₃, and that inhibitors of Src abolished As₂O₃ induced phosphorylation of and hence Cbl binding to EGFR. These findings implicate Src as a downstream effector of As₂O₃.

In carcinogenicity studies, arsenic induced activation of other signaling pathways is regarded to promote cellular proliferation (Yang and Frenkel, Environ Pathol Toxicol Oncol., 21(4):331-42 (2002)). The data also showed that only ERK1/2 phosphorylation was activated. However, this did not lead to a demonstrable biologic effect, as ERK inhibitor neither increased or decreased As₂O₃ induced cytotoxicity. JNK was activated as well, although the biologic effects are not immediately obvious in the current cell model. In leukemia cells, activation of As₂O₃-mediated JNK activation has been reported to contribute to induction of apoptosis (Davison, et al., Blood, 103(9):3496-502 (2004)).

Collectively the data show a new biological and therapeutic mechanism of controlling the EGFR signaling pathway. As₂O₃-induced EGFR degradation is reminiscent of the action of arsenic on another fusion oncoprotein PML-RARA in APL (Sternsdorf, et al., Mol Cell Biol, 19(7):5170-8 (1999); Lallemand-Breitenbach, et al., J Exp Med., 193(12):1361-71 (2001)). Previous studies had shown that As₂O₃ triggered conjugation of the ubiquitin-related peptide SUMO-1 to the PML part of PML-RARA. Sumolated PML-RARA is then sorted to the proteosome for degradation. In fact, As₂O₃-induced oncoprotein degradation had been proposed to be one of the important anti-cancer effects of As₂O₃. The data in the Examples show that As₂O₃ increased the ubiquitination of a growth factor receptor related to cancer cell survival and proliferation. These data provide a new paradigm whereby arsenic might mediate its anti-proliferative effect in cancer cells.

As₂O₃ is in general inhibitory to the growth of most cell lines in vitro. However, with cell lines commonly used in carcinogenicity studies (Qian, et al., J Inorg Biochem., 96(2-3):271-8 (2003)). Arsenic has been found to be stimulatory and might therefore contribute to carcinogenesis. As shown herein, activation of Src and EGFR might be construed biochemically to be carcinogenic. However, the ultimate biologic effect was suppression of cellular proliferation.

As₂O₃ is a new strategy of EGFR targeting. The therapeutic use of As₂O₃ is remarkably safe, side effects being usually minor. With the development of oral As₂O₃, cardiac QT prolongation is much reduced, so that arrhythmias are no longer a serious threat (Siu, et al., Blood, 108(1):103-6 (2006)). Treatment doses of As₂O₃ lead to plasma concentrations of 2-5 μM, well within the IC₅₀ of most cell lines tested in vitro (Kumana, et al., Eur J Clin Pharmacol, 58(8):521-6 (2002)). As₂O₃ is not ligand specific, so that it does not have the tissue precision of anti-EGFR antibodies. However, it is relatively inexpensive and can be used for prolonged durations.

B. Cytokine Dependent Cancers

Another embodiment provides a method for treating cancer dependent on cytokines or activation of cytokine receptors by administering an effective amount of As₂O₃ to down regulate the cytokine receptor, preferably IL-6R, in the cancer cell. Preferably, the cytokine receptor is down regulated post-translationally. The data provided herein demonstrate that As₂O₃ suppressed the growth of the multiple myeloma (MM) cell line U266 by inducing apoptosis. Furthermore, As₂O₃ totally abrogated the stimulatory effects of IL-6 and IGF-1 on U266. As₂O₃ inhibited the major effector pathways of IL-6, including JAK/STAT, Akt and Erk1/2. The simultaneous suppression of several different pathways meant that a common upstream component of IL-6 signaling cascade was involved, suggesting that the IL-6R might be targeted.

It was further established that As₂O₃ treatment led to a dose and time dependent suppression of the IL-6 complex, including the IL-6Rα and gp130, Not only was IL-6Rα and gp130 down-regulated, the interaction of the two proteins was also decreased. The transcription of the IL-6Rα and gp130 genes was unaffected. These results implied that down-regulation of the TL-6R (IL-6Rα and gp130) occurred at the post-transcription level.

As₂O₃ induced down-regulation of the surface IL-6Rα expression. The down-regulation was shown to be due to an internalization of the TL-6Rα protein in the myeloma cells. This was demonstrated by immunofluorescence studies and confirmed by flow cytometry. These results therefore suggest that As₂O₃ induced down-regulation of the surface IL-6Rα might be mediated by internalization of the IL-6R complex, leading to degradation in the lysosome or proteasome.

As₂O₃ induced IL-6Rα and gp130 down-regulation and internalization by increasing the binding of ubiquitin to these two proteins. The increase in ubiquitin binding to these proteins formed the first important step in their internalization and degradation (Marmor and Yarden, Oncogene, 23(11):2057-70 (2004)). This was an important novel finding, as the ubiquitination of IL-6Rα and gp130 has not been previously examined. Furthermore, pharmacological means of increasing IL-6Rα and gp 130 ubiquitination has never been described before.

As₂O₃-induced down-regulation of IL-6Rα was alleviated by the lysosomal inhibitor NH₄Cl. Furthermore, the proteasome inhibitor MG132 partly alleviated the suppressive effect of As₂O₃ on IL-6Rα. These results confirmed that As₂O₃ targeted the IL-6R complex to degradation in the lysosome and maybe also the proteasome.

As₂O₃ kills myeloma cells by targeting and down-regulating the interleukin-6 receptor. The destruction of the cancer cells is a consequence of degradation of a growth factor receptor, by a common mechanism of increased ubiquitination, leading to degradation in the lysosome and proteasome.

Importantly, both growth factor receptors (epidermal growth factor receptor and interleukin-6 receptor) are receptor tyrosine kinases. Thus, As₂O₃ targets receptor tyrosine kinases by increasing their ubiquitination and therefore degradation in the lysosome and proteasome.

Arsenic trioxide can be used for the treatment of cancers that are dependent on TRKs for proliferation, survival, metastasis and differentiation.

Patients with cancers that are dependent on TRKs for proliferation, survival, metastasis and differentiation can be treated with As₂O₃. Representative cancers include, but are not limited to head and neck squamous cell carcinoma (HNSCC) and multiple myeloma.

The dose of oral As₂O₃ is typically adjusted according to age and kidney function. In one embodiment, the dose range of As₂O₃ varies from 1 to 10 mg, typically about 5 to 10 mg.

The present invention will be further understood by reference to the following non-limiting examples.

### EXAMPLES RELATED TO EGFR

### Methods and Materials

Cancer Cell Lines.

The hypopharyngeal SCC cell line FaDu (Catley, et al., Drug Resist Updat., 8(4):205-18 (2005)) was maintained in Eagle's minimum essential medium (MEM; Invitrogen Corporation, Carlsbad, Calif., USA) supplemented with 10% fetal bovine serum in 5% CO₂ at 37° C.

MTT Assay.

Cellular proliferation was assessed by an MTT assay (GE Healthcare, Piscataway, N.J., USA). Briefly, cells (5,000 cells/well in 90-well plates) were incubated for 48 hours with control medium or reagents. MTT assays were then performed by incubating the treated cells with the MTT labeling solution (0.5 mg/ml). After 4 hours of incubation, cells were lysed, formazan crystals solubilized and detected at 570 nm. All experiments were performed in triplicate.

Annexin V Apoptosis Assay

Cells were trypsinized, washed and incubated on ice for 10 minutes with FITC-conjugated annexin-V and propidium iodide (PI) (Immunotech; Fullerton, Calif., USA). Apoptotic cells (annexin-V-positive, PI-negative) were enumerated by flow cytometry (Epics, Beckman Coulter, Fullerton, Calif., USA) after appropriate color compensation.

Western Blot Analysis.

Western blot analysis was performed as previously reported. Briefly, cell lysis and collection of protein was conducted according to standard protocols. Protein samples (typically 20 μg) were separated by SDS-PAGE in 9% or 12% resolving gel and electro-transferred to nitrocellulose membranes. Incubation with the primary antibody was performed overnight at 4° C., followed by reaction with 1:1,500 horseradish peroxidase-conjugated secondary antibodies (Amersham-Pharmacia Biotechnology, Piscataway, N.J., USA). Immunoreactive bands were detected with either the Western Lightning Chemiluminescence reagent (PE Biosystems, Foster City, Calif., USA), or the SuperSignal West Pico Chemiluminescent Substrate (Pierce Chemical Co., Rockford, Ill., USA), and visualized on X-ray film. Integrated density of immunoreactive bands was quantified by the BioChemi gel documentation system (UVP Inc., Upland, Calif., USA). All experiments were performed in triplicates.

Immunoprecipitation.

Immunoprecipitation was performed by standard procedures Briefly, cells were washed with ice-cold phosphate-buffered saline (PBS) supplemented with 1 mM sodium orthovanadate and Complete protease inhibitor cocktail (Complete; Roche Molecular Biochemicals), lysed with lysis buffer (50 mM Tris-Cl, pH; 8, 150 mM NaCl, 1% TX, 1× aprotinin (Sigma, St Louis, Mo., USA), 1 mM phenylmethylsulfonyl fluoride, 1 mM sodium orthovanadate, and 1× Complete) at 4° C. for 15 minutes. Lysates were collected and centrifuged. Protein assays were performed with the Bio-Rad Protein Assay Kit (Philadelphia, Pa., USA), and adjusted to 1 μg/μL (typically 800 to 1000 μg). Immunoprecipitation was preformed by incubation of protein samples with the appropriate antibodies (typically 4 μg) at 4° C. overnight with general shaking. For each reaction, the antibody/protein complex was precipitated by incubation of 30 μL of Protein G beads (Zymed, San Francisco, Calif., USA) at 4° C. for 2 hour with general shaking. Protein C beads were then washed 3 times with 400 μL of lysis buffer. The supernatant was aspirated, and 50 μL of 2× Laemmli buffer was added. The antibody/protein complex was released from the beads by heating at 95° C. for 10 minutes. The immunoprecipates were then analyzed by Western blotting.

Reagents and Antibodies.

Reagents used included As₂O₃ and dimethylsulfoxide (Sigma, St Louis, Mo., USA); EGFR inhibitor AG1478 (Biosource, Camarillo, Calif., USA); Src inhibitors PP1, PP2 and PP3 (Calbiochem, Merck, Darmstadt, Germany). Antibodies used included anti-EGFR neutralizing antibody (clone LA1, Upstate Biotechnology, Lake Placid, N.Y., USA); rabbit anti-EGFR anti-sera and rabbit anti-cbl anti-sera (Santa Cruz Biotechnology, Santa Cruz, Calif., USA); mouse monoclonal anti-ubiquitin anti-sera, mouse monoclonal anti-phosphorylated tyrosine anti-sera, mouse monoclonal anti-STAT3 antibody, rabbit anti-phosphorylated EGFR (tyrosine 1045) (pEGFR-1045) anti-sera, rabbit anti-phosphorylated STAT3 (tyrosine 705) (Py-STAT3) anti-sera, mouse monoclonal anti-caspase-3 antisera, rabbit polyclonal anti-phosphorlyated Erk1/2 (p-Erk1/2) anti-sera, mouse monoclonal anti-Erk-2 anti-sera, rabbit polyclonal anti-phosphorylated p54/p46 JNK anti-sera, rabbit polyclonal anti-Src anti-sera, rabbit polyclonal anti-phosphorylated Src (tyrosine 416, p-Src-416) (Cell Signaling Technology, Beverly, Mass., USA); rabbit polyclonal anti-phosphorylated Akt (p-Akt) and anti-Akt anti-sera (Pharmingen, B D Biosciences, San Jose, Calif. USA), mouse anti-actin antibody, (Sigma); mouse monoclonal anti-hemaglutinin (HA) antibody (Zymed).

Plasmids and Primers.

Plasmid encoding the full-length wild type EGFR in pUSEamp vector (pUSEamp-EGFR) was obtained from Upstate. Primers for reverse-transcription polymerase chain reaction (RT-PCR) of EGFR were F₁ (forward: 5′-TGA CTC CTT CAC ACA TAC TC-3′ (SEQ ID NO:1) corresponding to bp 1320-1339 in exon 10, accession number: AF288738) and R₁ (reverse: 5′-TTC TCA AAG OCA TGG AGG TC-3′ (SEQ ID NO:2) corresponding to bp 1432-1451 in exon 11). Primers and probes for quantification of EGFR mRNA by quantitative PCR (Q-PCR) were Q_{F} (forward: 5′-GAC AGC ATA GAC GAC ACC TTC CT-3′ (SEQ ID NO:3), corresponding to bp 3238-3260 in exon 27), Q_{R} (5′-GCC AGC GGG CCT TTT G-3′ (SEQ ID NO:4), corresponding to bp 3294-3309 in exon 28) and the TaqMan probe Q_{T} (5′-CCA GTG CCT GAA TAC ATA AAC CAG TCC GTT C-3′ (SEQ ID NO:5), corresponding to bp 3262-3292 crossing exon 27 and 28, and dual-labeled at the 5′ end with 6-carboxyfluorsecein, FAM, and the 3′ end with 6-carboxytetramethylrhodamine, TAMRA). Primers and TaqMan probe were designed by the Primer Express software (PE Biosystems).

The following plasmids were be used in transfection experiments: pUSEamp-EGFR (containing the full length EGFR); p-HA-Ub (containing wildtype ubiquitin tagged to hemagglutinin); p-HA-Ub-GG (containing a mutant ubiquitin that precluded substrate binding); and p-OFP (containing green fluorescence protein).

RNA and PCR.

Total RNA was prepared using TRIzol (Invitrogen, Carlsbad, Calif., USA). Quantitative-PCR was performed with the ABI Prism 7700 Sequence Detector (PE Biosystems) as reported (Roodman, Blood Cells Mol. Dis., 32(2):290-2 (2004)). Q-PCR was set up in a reaction volume of 50 μL using the TaqMan Universal PCR Master Mix (PE Biosystems), containing 10 μL cDNA, 200 and 400 nM Of Q_{F} and Q_{R}, and 50 nM QT. Thermal cycling was initiated with a 2-minute incubation at 50° C., followed by a first denaturation step of 10 minutes at 95° C., and then 40 cycles of 95° C. for 15 seconds and 60° C. for 1 minute. Q-PCR data were collected and analysed by the ABI Sequence Detection System.

Transfection.

Transfection of cancer cells was performed by using Lipofectamine 2000 reagent (Invitrogen). Target cells were seeded at about 80% to 90% confluence in 60 mm tissue culture plate and incubated for 24 hours. On the day of transfection, 6 μg of each plasmid was diluted with 500 μL of serum-free MEM in separated tubes. For each transfection, 40 μL of Lipofectamine 2000 reagent was diluted to 500 μL serum-free MEM in a separated tube and incubated at room temperature for 5 minutes. After 5 minutes of incubation, each diluted plasmid was mixed with 500 μL diluted Lipofectamine 2000 reagent and incubated in room temperature for 20 minutes, leading to the formation of a DNA/Lipofectamine 2000 complex. After 20 minutes of incubation, the complex was added to cells and incubated at 37° C. for 48 hours. Cells were then selected with 50 μg/ml G418.

### Example 1

### As₂O₃ Induced Apoptosis in FaDu Cells by Activation of Caspase-3

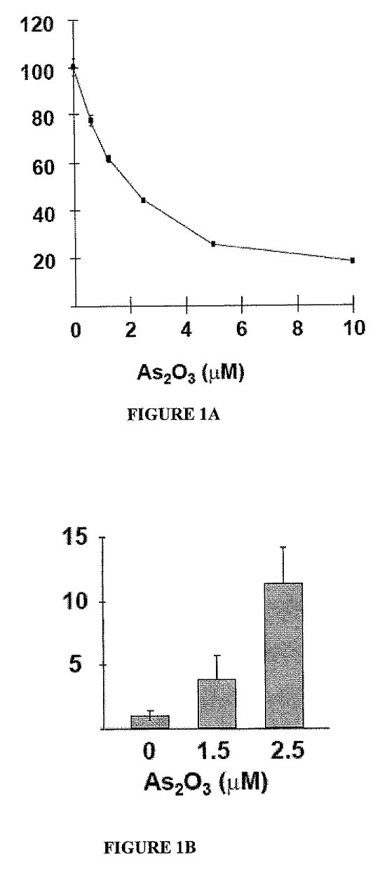

MTT results showed that As₂O₃ inhibited FaDu cell proliferation in a dose- and time-dependent manner (FIG. 1A). After 2 days of As₂O₃ treatment at 2.5 μM, about 60% of FaDu cells were apoptotic, as determined by flow cytometric analysis of annexin-V/PI. Western blot analysis showed that activated (cleaved) caspase-3 was progressively up-regulated with increasing concentrations of As₂O₃ (FIG. 1B), suggesting that the apoptosis was mediated via the caspase 3 pathway.

### Example 2

### EGFR Signaling was Necessary for FaDu Cell Proliferation and Targeted by As₂O₃

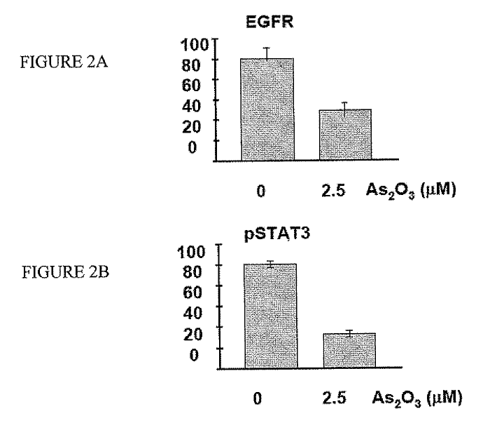

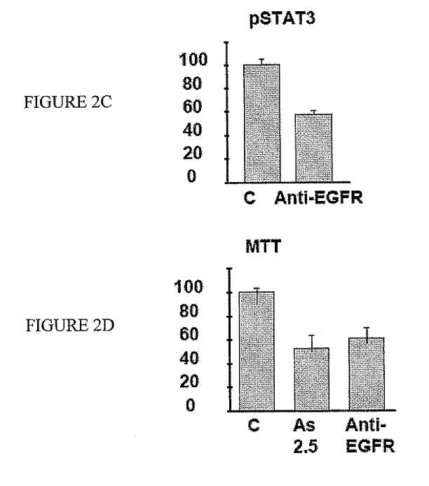

Western blot analysis showed that FaDu cells expressed high levels of EGFR (FIG. 2A). Therefore, EGFR signaling might be involved in FaDu cell proliferation. Indeed, there was constitutive phosphorylation of STAT3, a downstream effector of EGFR signaling. Western blot analysis showed that EGFR was targeted by As₂O₃. Treatment of FaDu cells with As₂O₃ led to down-regulation of EGFR and hence decreased phosphorylation of STAT3 (FIG. 2B). To show that EGFR signaling was critical for cellular proliferation, FaDu cells were treated with a neutralizing anti-EGFR antibody. This led to suppression of STAT3 phosphorylation (FIG. 2C), which resulted in a decrease of cellular proliferation to an extent comparable with As₂O₃ treatment (FIG. 2D). These results suggested that As₂O₃ inhibited FaDu cell proliferation by targeting EGFR. RT-PCR showed that FaDu cells constitutively expressed EGF. However, the EGF transcription was not affected by As₂O₃ treatment.

### Example 3

### As₂O₃ Did not Affect EGER Gene Transcription

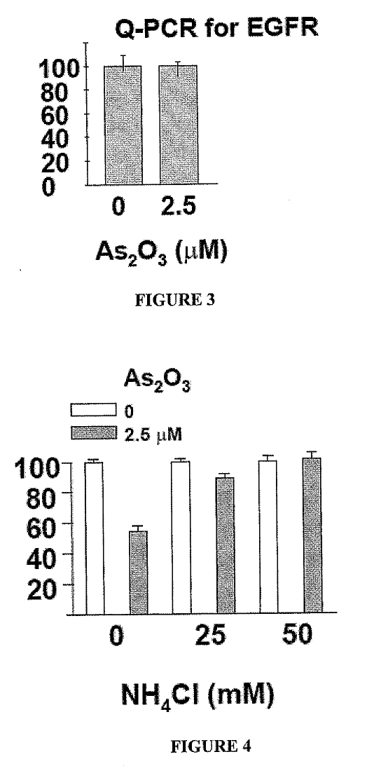

Semi-quantitative RT-PCR showed that As₂O₃ treatment did not affect EGFR gene transcription. The results were confirmed by Q-PCR, which showed practically no change in EGFR mRNA before and after As₂O₃ treatment (FIG. 3). Therefore, As₂O₃ down-regulated EGFR by post-transcriptional mechanisms, possibly by increasing its degradation.

To investigate the reason for As₂O₃ induced down-regulation of EGFR, Western blot analysis was performed. Triplicate experiments showed a dose dependent increase of EGFR phosphorylation at tyrosine 1045 with As2O3 treatment (1 hour). There was yet no change in EGFR at this time point. Immunoprecipitation with an anti-EGFR antibody followed by immunoblotting with the appropriate antibodies was performed. There was a dose dependent increase in Cbl binding with As₂O₃ treatment (1 hour), in parallel with increase in EGFR 1045 phosphorylation. Immunoprecipitation with an anti-EGFR antibody was followed by immunoblotting with the appropriate antibodies. As₂O₃ (100 μM for 1 hour) led to increased EGFR phosphorylation and Cbl binding, which was totally abrogated by pre-treatment with AG 1478 (AG) (for 1 hour). These results were comparable with those observed after treatment with EGF (10 μM for 1 hour). Further experiments of the effects of As₂O₃ on EGFR ubiquitintion were performed. Immunoprecipitation with an anti-EGFR antibody followed by immunoblotting with appropriate antibodies was performed. Treatment with As₂O₃ (100 μM for 1 hour) led to a significant increase in ubiquitination of EGFR as compared with control, suggesting that As₂O₃ acts by increasing EGFR ubiquitination.

To further demonstrate that Src may be involved in As₂O₃ actions, As₂O₃ induced EGFR phosphorylation and Cbl binding was investigated by Western blot analysis for the relationship of Src and its inactivation by pretreatment (for 1 hour) with Src-specific inhibitors PP1 and PP2 (but not the inactive analogue PP3), on As₂O₃ (100 μM for 1 hour)-induced EGFR tyrosine 1045 phosphorylation. Inhibition of Src prevented As₂O₃ induced EGFR phosphorylation. Immunoprecipitation with an anti-EGFR antibody was followed by immunoblotting with appropriate antibodies. Src inhibitors PP1 and PP2 decreased As₂O₃-induced EGFR tyrosine phosphorylation and abrogated Cbl binding. The inactive PP3 analogue did not suppress EGFR phosphorylation, but abrogated Cbl binding because of an inherent EGFR inhibiting activity.

### Example 4

### As₂O₃ Induced EGFR Tyrosine 1045 Phosphorylation and Consequently c-cbl Binding

More than 50% of EGFR had been down-regulated 48 hours post-As₂O₃ treatment. To evaluate the initial events leading to EGFR down-regulation, early time points had to be investigated. FaDu cells were therefore examined one hour after As₂O₃ treatment. EGFR phosphorylation at tyrosine 1045 is the first step in its degradation. Indeed, As₂O₃ treatment resulted in a dose-dependent increase in EGFR phosphorylation at tyrosine 1045. EGFR tyrosine 1045 phosphorylation provided a docking site for Cbl binding, which was confirmed by immunoprecipitation showing a dose dependent increase in Cbl binding to EGFR with As₂O₃ treatment.

### Example 5

### As₂O₃ Induced Cbl Binding to EGFR is Phosphorylation Dependent

Treatment of FaDu cells with the tyrosine kinase inhibitor AG 1478 abolished As₂O₃ induced EGFR tyrosine phosphorylation. Expectedly, As₂O₃ induced Cbl binding to EGFR was also abrogated. The molecular events of As₂O₃ treatment were comparable with those consequent to stimulation of EGFR by EGF, which would initiate its degradation. These observations suggested that As₂O₃-induced EGFR phosphorylation and hence Cbl binding might be a process critical for the down-regulation of EGFR.

### Example 6

### As₂O₃ Induced EGFR Degradation by Ubiquitination

To demonstrate that As₂O₃-enhanced Cbl binding to EGFR resulted in ubiquitination, FaDu cells were transiently transfected with the plasmid p-HA-Ub, and then treated with As₂O₃. Immunoprecipitation showed that As₂O₃ treatment led to a significant increase in EGFR ubiquitination.

### Example 7

### As₂O₃ Suppression of EGFR was Lysosome Dependent

To show that ubiquitinated EGFR was degraded by the lysosome, FaDu cells were pre-incubated with the lysosome inhibitor ammonium chloride (NH₄Cl), and then treated with As₂O₃. The results showed that NH₄Cl treatment reversed As₂O₃-mediated suppression of EGFR, confirming that EGFR down-regulation was lysosomal dependent (FIG. 4).

### Example 8

### As₂O₃ Induced EGFR Phosphorylation was Related to Src Activation

To explore the mechanism of As₂O₃-induced EGFR phosphorylation, mediators of EGF-independent signaling pathways were examined. The non-receptor tyrosine kinase Src has been shown to mediate EGFR phosphorylation. In FaDu cells, Src was constitutively phosphorylated. As₂O₃ treatment led to a further increase in Src phosphorylation. Treatment with the Src inhibitors PP1 and PP2 suppressed Src phosphorylation. Interestingly, this was associated with abrogation of As₂O₃-induced EGFR tyrosine 1045 phosphorylation. The inactive analogue PPS was ineffective in suppressing Src phosphorylation, which resulted in preservation of As₂O₃-induced EGFR tyrosine 1045 phosphorylation. Immunoprecipitation confirmed that inhibition of Src by PP1 and PP2 led to inhibition of As₂O₃-induced EGFR-1045 phosphorylation, which expectedly resulted in inhibition of Cbl binding to EGFR. The inactive analogue PP3 was ineffective in suppressing As₂O₃-induced EGFR tyrosine 1045 phosphorylation. However, PP3 is itself a known EGFR inhibitor, which might account for the inhibition of Cbl binding despite preservation of EGFR tyrosine 1045 phosphorylation.

### Example 9

### As₂O₃ Did not Target Other EGFR Signaling Pathway Effectors

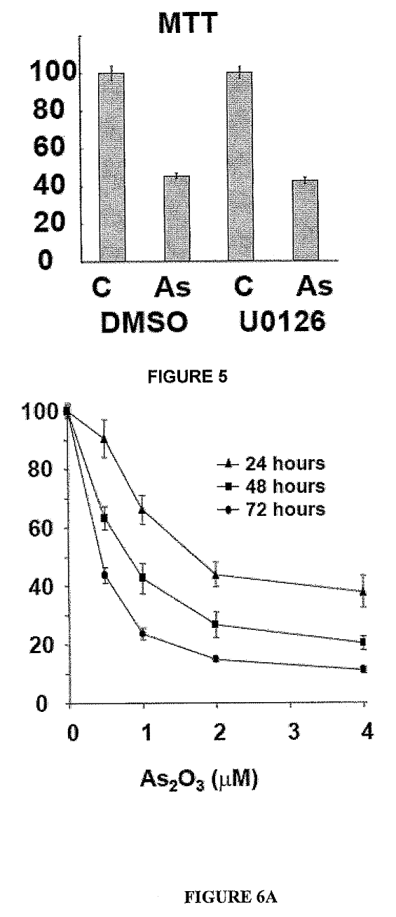

To examine if As₂O₃ affected other components of EGFR signaling, downstream effectors of the pathway were studied. As₂O₃ did not affect Akt phosphorylation. Erk1/2 phosphorylation was increased with As₂O₃ treatment. However, treatment with the Erk inhibitor U0126 had no effect on As₂O₃ cytotoxicity (FIG. 5), suggesting that activation of Erk was not a major downstream mechanism of As₂O₃ cytotoxicity. As₂O₃ also induced JNK phosphorylation, which was not associated with EGFR signaling. In contrast to EGFR, the increase phosphorylation of Erk1/2 and JNK was unrelated to Src.

### EXAMPLES RELATED TO TL-6R

### Methods and Materials

Cell Line.

The human myeloma cell line U266 was obtained from American Type Culture Collection (Manassas, Va., USA). Cells were maintained in RPMI1640 (Invitrogen, Carlsbad, Calif., USA) supplemented with 10% fetal bovin serum (Invitrogen),

Reagents and Antibodies.

Reagents and antibodies used included As₂O₃ and ATRA (Sigma, St Louis, Mo., USA); bortezomib (Millennium, Cambridge, Mass., USA); lysosome inhibitor ammonium chloride (NH₄Cl, AMRESCO, Solon, Ohio, USA); proteosome inhibitor M0132, p38 inhibitor SB203580, AK inhibitor SP600125, MEK inhibitor U0126 and pan-caspase inhibitor Z-VAD-FMK (Calbiochem, La Jolla, Calif., USA); rabbit polyclonal antibodies to total (and phosphorylated) JAK2 (Tyrosine-10007/1008), STAT3 (Tyrosine-705), ERK1/2 (Threonine-202/Tyrosine-204), Akt (Serine-743), p38 (Threonine-180/Tyrosine-182) and AK (Threonine-183/Tyrosine-185) (Cell signaling Technology, Beverly, Mass., USA); rabbit polyclonal antibodies to caspase-3, NF-κB, cyclin D1 and phosphorylated tyrosine (Cell signaling Technology); rabbit polyclonal antibodies to IL-6Rα (C20) and gp130 (Santa-Cruz, Sant Cruz, Calif., USA); rabbit polyclonal antibody to survivin (Calbiochem); mouse monoclonal neutralizing antibody against IL-6Rα R & U Systems, Minneapolis, Minn., USA); mouse monoclonal antibodies to ubiquitin, clone FK1 and FK2 (Biomol, Plymouth Meeting, Pa., USA) and to actin (Sigma); horseradish peroxide (HRP)-conjugated goat anti-rabbit immunoglobulin G (IgG) and rabbit anti-mouse IgG secondary antibodies (Zymed, South San Francisco, Calif., USA); and fluorescent-conjugated secondary antibody, Alexa Fluor® F(ab′)₂ fragment of goat anti-rabbit IgG antibody (Molecular Probes, Eugene, Oreg., USA).

Cell Proliferation Assay.

Cell proliferation was analyzed by the MTT assay. Cells (3×10⁴) were cultured in 96-well plates with the tested drugs, after which 10 μl of 5 mg/ml MTT per well was added. Four hours later, solubilizing buffer (100 μl/well) was added to dissolve the formazan crystal, followed by determination of absorbance at 570 nm. All experiments were performed in quadruplicates.

Flow Cytometric Analyses.

For evaluation of apoptosis, 1×10⁶ cells were treated with As₂O₃ (4 μM, 0-8 hours), washed with ice-cold phosphate-buffered-saline (PBS) and resuspended in binding buffer containing annexin V-FITC and propidium iodide (PI) (Beckman Coulter, Paris, France) for 15 minutes in dark. Apoptotic cells (annexin-V positive, popidium iodide negative) were then analyzed. For expression of IL-6Rα, 1×10⁶ cells were suspended in 100 μl of PBS containing 20 μl of PE-conjugated anti-CD126 (IL-6Rα) antibody, incubated in dark for 20 minutes, and then analyzed for CD126 expression. All analyses were performed on an Epics flow cytometer (Beckman Coulter).

Western Blot Analysis.

Cells were harvested by centrifugation, washed with ice-cold PBS, and lysed with radioimmunoprecipitation buffer (RIPA) supplemented with protease inhibitors (1 mg/ml aproptinin, lupeptin, and pepstatin, 1 mM phenylmethysulfony fluoride), phosphatase inhibitors (1 mM sodium orthovanadate, 1 mM sodium fluoride), and protease inhibitor mix (Complete; Roche Diagnostic, Alameda, Calif., USA) at 4° C. for 20 minutes. Lysates were vortexed for 20 seconds thrice on ice and centrifuged at 14,000 r.p.m. for 10 minutes. Protein concentration was determined by the Bio-Rad Protein Assay Kit (Bio-rad, Hercules, Calif., USA). Total cell lysate was mixed with one-forth volume of 5× Laemmli sample buffer and heated at 95° C. for 10 minutes. Proteins were resolved by sodium dodecylsulfate-polyacrylamide gel electrophoresis (SDS-PAGE), electro-transferred to nitrocellulose membrane (PE Biosystems, Foster City, Calif., USA) (100V, 2 hours), and blocked by 5% non-fat milk in TBS-Tween for 1 hour. Membranes were incubated with primary antibody at 4° C. overnight, followed by horseradish peroxidase-conjugated secondary antibodies (Zymed) for 90 minutes. After washing, immuno-reactive protein bands were detected using Western Lightning Chemiluminescence reagent (PE Biosystems), and visualized after exposure on X-ray film (Fuji, Tokyo, Japan).

Immunoprecipitation.

To immunoprecipitate IL-6Rα and gp130, total cell lysate was prepared from 1×10⁷ cells. Cell lysate was incubated at 4° C. with 4 μg of specific antibody to IL-6Rα or gp130 in 500 μl of RIPA buffer/inhibitors for 2 hours. Rec-Protein G sepharose 4B solution (40 pt) (Zymed) was added after 3 washes with 500 μl of ice-cold RIPA buffer/inhibitors. Following further incubation for 90 minutes at 4° C., beads were collected by centrifugation and washed four times with 1 ml of ice-cold RIPA buffer/inhibitors. Washed beads were then resuspended in 30 μl of 2× Laemmli sample buffer and immunoprecipitated protein was eluted by heating at 95° C. for 5 minutes.

Semi-Quantitative and Quantitative Polymerase Chain Reaction (PCR).

Total mRNA was prepared with TRIZOL™ (Invitrogen) by standard procedures. Reverse transcription was performed with 1 μg RNA by M-MLV reverse transcriptase (Invitrogen). PCR was performed with one tenth of the cDNA with 2.5 U of Fast-Taq polymerase (Roche) in 1×PCR Buffer, 2.5 mM MgCl₂, 0.2 μM of forward/reverse primers, and 250 μM of each of the four dNTP, at a final volume of 25 μl in a thermal cycler (9700, PE Biosystems). Real-time quantitative PCR (Q-PCR) was performed with the Assays-on-Demand™ Gene Expression Products (AssayID: Hs00794121_ml) (PE Biosystems) according to the manufacturer's instructions (ABI Prism 7700 Sequence Detector, PE Biosystems). The β-actin was used as an internal control for cDNA input. Sequence of primers and reaction conditions were shown in table 1. All experiments were performed in triplicates.

Cloning of IL-6Rα Expression Plasmid.

The complete coding sequence of IL-6Rα (Gene Bank accession: NM000565) was amplified from total RNA of peripheral blood cells from a normal donor (Table 1), and cloned into the BamHI and EcoRI sites of the pcDNA3.1(+) mammalian expression vector (Invitrogen) to give pcDNA3.1-IL-6Rα. Nucleotide sequence of the cloned IL-6Rα fragment was confirmed by DNA sequencing.

Transfection.

pc-DNA3.1-IL-6Rα was transfected into U266 cells by electroporation. Cells (5×10⁶) were mixed with 20 μg of vector in 300 μl of serum-free medium, and subjected to electroporation (250V, 960 μF) (Bio-Rad Gene Pulser X-Cell™, Bio-Rad). U266 cells stably over-expressing IL-6Rα (U266-IL-6Rα) was selected and maintained in G418 (1 mg/ml).

Immunofluorence Staining and Confocal Microscopy.

Sterilised coverslips were immersed in poly-L-Lysine (PLL) solution (10 mg/ml) for 20 minutes, rinsed with PBS, and placed into 6-well culture plate (I coverslip/well). U266-IL-6Rα cells were cultured onto PLL-coated coverslips at a density of 5×10⁵ cells/ml overnight Cells were then treated with As₂O₃ (4 μM, 0-8 hours). Culture medium was removed, coverslips rinsed once with ice-cold PBS, cells fixed with 100% methanol (−20° C.) for 10 minutes and air-dried. After blocking with 10% goat serum and washing, coverslips were incubated overnight at 4° C. with primary antibody to IL-6Rα. Coverslips were then washed with PBS, and incubated at room temperature for 45 minutes in the dark with the Alexa Fluor® 488 F(ab′)₂ antibody. After thorough washing, the coverslips were mounted onto glass slides with Clearmount mounting solution (Zymed) and examined under confocal microscopy.

### Example 10

### As₂O₃ Decreased U266 Cellular Proliferation in Response to IL-6/IGF-1 and Induced Apoptosis

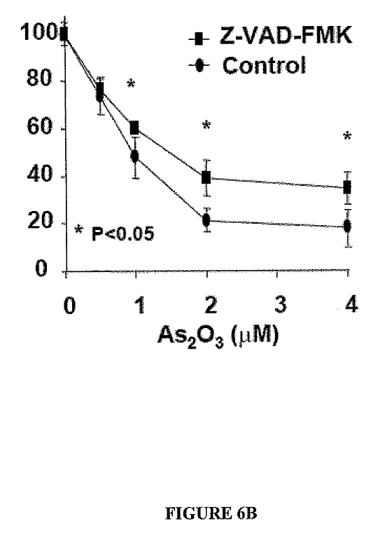

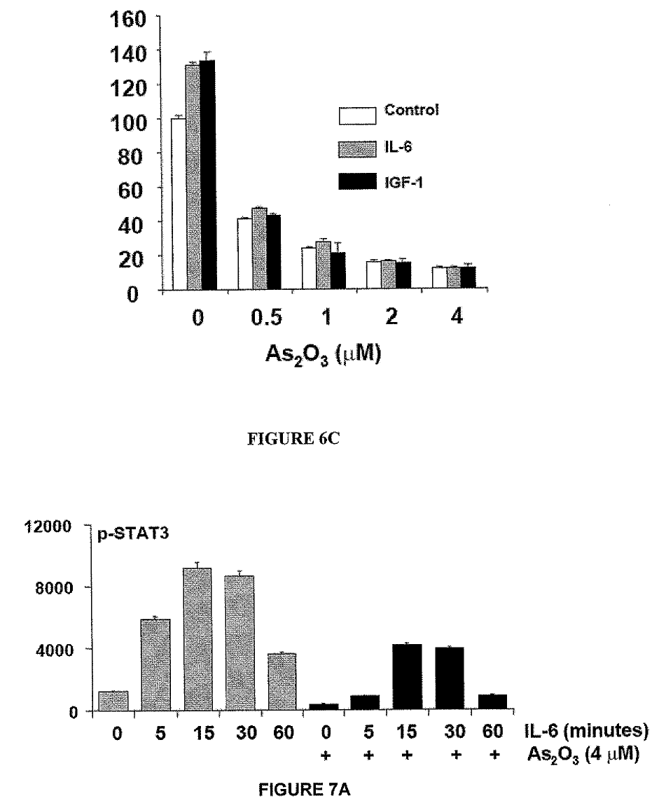

As₂O₃ treatment of U266 led to a dose and time dependent suppression of cellular proliferation (FIG. 6A). Although U266 cells grew autonomously, they still responded to additional stimulation by IL-6 and IGF-1. However, As₂O₃ totally negated the stimulatory effects of IL-6 and IGF-1. Flow cytometric analysis showed that As₂O₃ treatment resulted in apoptosis. Western blot analysis showed a dose and time dependent up-regulation of activated caspase 3 after As₂O₃ treatment. However, pre-treatment with the pan-caspase inhibitor Z-VAD-FMK only partly rescued U266 cells from As₂O₃ cytotoxicity (FIG. 6B). These results suggested that As₂O₃ inhibited U266 cell growth by both caspase dependent and independent mechanisms.

### Example 11

### As₂O₃ Suppressed the IL-6 Signaling Pathway

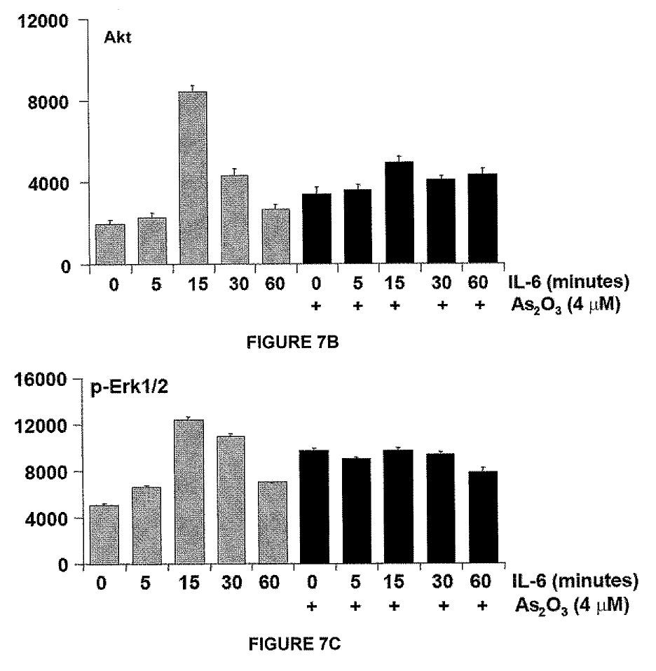

The effects of As₂O₃ on major signaling pathways in U266 cells as induced by IL-6 were examined. Treatment with IL-6 (50 ng/ml) induced the phosphorylation of JAK2, STAT3, Akt and ERK1/2, which were known downstream effectors of IL-6. Pre-treatment of U266 cells with As₂O₃ (4 μM) significantly suppressed subsequent IL-6 enhanced phosphorylation of JAK2, STAT3, Akt and ERK1/2 (FIGS. 6C, 7A-C). These results implied that As₂O₃ might inhibit IL-6 signaling by blocking components upstream of these pathways, possibly the IL-6R.

### Example 12

### A₂O₃ Targeted IL-6R

To test whether As₂O₃ targeted the IL-6R, U266 cells were pre-treated with either an IL-6R neutralizing antibody (110 μg/mL) or As₂O₃ (4 μM) for 4 hours, and then stimulated with IL-6. Both the anti-IL-6R neutralizing antibody and As₂O₃ totally suppressed IL-6 induced STAT3 phosphorylation, implying that As₂O₃ might target the IL-6 receptor. Western blot analysis showed that As₂O₃ induced a dose and time dependent down-regulation of gp 130 and IL-6Rα. At 4 μM, As₂O₃ decreased gp130 and IL-6Rα at as early as 2 hours, and after 8 hours, both proteins were almost completely down-regulated. The results suggested that As₂O₃ suppressed IL-6 signaling at the IL-6 receptor level. This was further confirmed by investigating the effect of As₂O₃ on IL-6R complex formation. U266 cells were treated with IL-6 with or without pre-incubation with As₂O₃. Cell lysates were then immunoprecipitated with an anti-gp 130 antibody. Subsequent immunoblotting showed that As₂O₃ inhibited IL-6-induced binding of IL-6Rα to gp130, leading to decreased phosphorylation of gp130. The results showed that As₂O₃ targeted both IL-6Rα and gp130.

### Example 13

### IL-6R was Suppressed by As₂O₃ Post-Transcriptionally

Semi-quantitative PCR showed no change in IL-6Rα and gp130 gene transcription. This was confirmed by real-time quantitative PCR, showing that As₂O₃ treatment did not lead to demonstrable change in IL-6Rα and gp130 mRNA. Therefore, As₂O₃ suppressed IL-6Rα and gp130 at a post-transcriptional level.

### Example 14

### As₂O₃ Induced Internalization of IL-6Rα

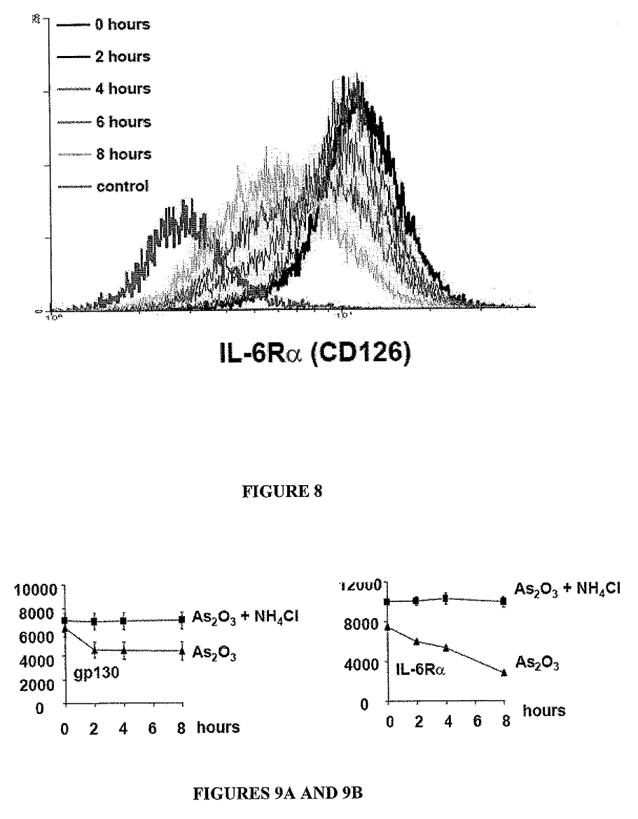

To examine the mechanism of down-regulation of IL-6R, U266 cells stably transfected pc-DNA3.1-IL-6Rα and hence over-expressing IL-6Rα (U266-IL-6Rα) were studied. Western blot analysis confirmed that U266-IL-6Rα over-expressed IL-6Rα by about 2,000 fold. Immunofluorescence studies under confocal microscopy showed that in untreated U266-IL-6Rα cells, IL-6Rα was detected predominantly at the cell surface. On treatment with As₂O₃ (4 μM), there was a time-dependent reduction in plasma membrane staining, with a redistribution of IL-6Rα intracellularly. Internalization of IL-6Rα was almost complete at 8 hours. These results were confirmed by flow cytometry, which also showed a time-dependent decrease in surface IL-6Rα expression with As₂O₃ treatment (FIG. 8). These observations suggested that As₂O₃ induced internalization of IL-6Rα.

### Example 15

### As₂O₃ Induced IL-6Rα and gp130 Ubiquination

The internalization of the IL-6R complex is partly mediated by ubiquitination. To study whether ubiquitination was involved in As₂O₃-induced down-regulation of IL-6R, U266 cells were incubated with 0-4 μM of As₂O₃, lysed, and subjected to immunoprecipitation with anti-IL-6Rα or anti-gp130 antibodies. Immunoblotting of the immunoprecipitates with an anti-ubiquitin antibody FK2, which recognized both mono- and poly-ubiquitin moieties, showed a dose dependent increase in ubiquitination of both IL-6Rα and gp130. These results confirmed that As₂O₃ induced the ubiquitination of the IL-6R complex, leading to its internalization and down-regulation. Internalization of IL-6Rα occurs after ligation with IL-6, but the molecular mechanisms controlling this process and the subsequent ubiquitination of IL-6α remains undefined (Hussein, et al., Br J Haematol., 125(4):470-6 (2004)). Internalization of gp130, however, may be related to phosphorylation of a serine residue at 782 (Ser-782), which is adjacent to a di-leucine internalization motif Western blot analysis, however, showed that As₂O₃ treatment did not lead to an increase in gp130 Ser-782 phosphorylation.

### Example 16

### As₂O₃ Induced Suppression of IL-6R was Blocked by Lysosomal Inhibition

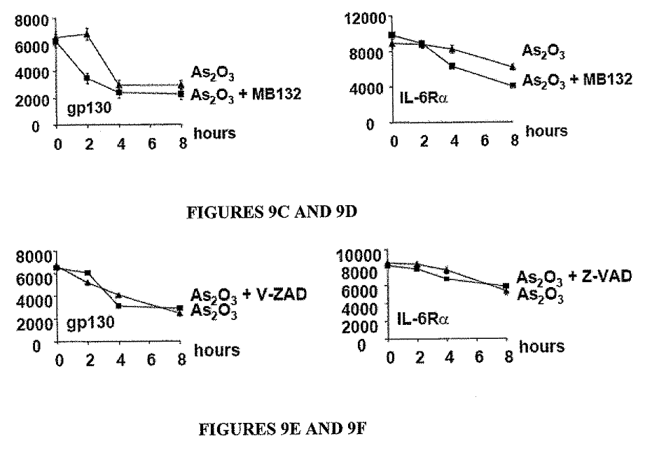

To investigate if As₂O₃-induced internalization and hence down-regulation of IL-6R was due to increased degradation, U266 cells were incubated with the lysosomal inhibitor ammonium chloride (NH₄Cl, 40 mM) or the proteosome inhibitor MG132 (25 μM) for 1 hour before As₂O₃ (4 μM) treatment. NH₄Cl prevented As₂O₃-induced suppression of IL-6Rα and gp130 (FIGS. 9A-B), whereas MG132 was ineffective (FIGS. 9C-D). As apoptosis was induced by As₂O₃ treatment, and caspase-3 might also mediate protein degradation, the effect of the pan-caspase inhibitor Z-VAD-FMK on As₂O₃-mediated IL-6Rα degradation was examined. The results showed that inhibition of caspase-3 had no effect on As₂O₃-mediated IL-6Rα and gp130 degradation (FIGS. 9E-F). These results confirmed that As₂O₃ suppressed the IL-6R complex by increasing its ubiquitination, hence targeting it to degradation in the lysosome.

### Example 17

### As₂O₃ Did not Affect IGF-1 Signaling

Treatment with IGF-1 induced the activation (phosphorylation) of IGFR and Akt. As₂O₃ treatment exerted no effect on IGFR and Akt phosphorylation, suggesting As₂O₃ might not target IGF-1 signaling. Finally, As₂O₃ treatment had no effect on IGF-1 receptor (IGF-1R) level. These results showed that although As₂O₃ suppressed IGF-1 induced stimulation of U266 cells, the inhibition was not mediated via targeting of the IGF-1 signaling pathway.

We claim:

1. A method for promoting cytokine receptor or receptor tyrosine kinase degradation in cancer cells comprising contacting the cells with an amount of arsenic trioxide effective to down regulate a cytokine receptor therein.

2. The method of claim 1 wherein the cell is from a cancer dependent on the receptor tyrosine kinase for development, progression, proliferation, differentiation or metastasis.

3. The method of claim 1, wherein the cancer is an EGFR dependent cancer.

4. The method of claim 3 wherein the cancer is a head and neck squamous cell carcinoma (HNSCC).

5. The method of claim 1 wherein the cancer is dependent on the cytokine receptor for development, progression, proliferation, differentiation or metastasis.

6. The method of claim 5 wherein the cancer is a multiple myeloma.

7. The method of claim 1, wherein the cytokine receptor is IL-6R.

8. The method of claim 1 wherein the arsenic trioxide is administered orally.

9. The method of claim 1 wherein the arsenic trioxide is in a pharmaceutically acceptable carrier for enteral administration.

10. The method of claim 1 wherein the arsenic trioxide is in a pharmaceutically acceptable carrier for parenteral administration.

11. The method of claim 1 wherein the arsenic trioxide is present in an amount from 5 to 10 mg.

12. The method of claim 10 wherein the arsenic trioxide is in a unit dosage form selected from the group consisting of solutions, suspensions, emulsions, syrups, tablets, and capsules.

13. The method of claim 1 wherein the arsenic trioxide is administered in an amount effective to produce a plasma concentration of between 2 and 5 μM.
